(19) United States
(12) Patent Application Publication
(10) Pub. No.: US 2003/0208219 A1
(43) Pub. Date: Nov. 6, 2003

(54) Steerable biliary catheter

(76) Inventors: Harold M. Aznoian, North Andover, MA (US); Michael LeBlanc, Bolton, MA (US)

Correspondence Address: KIRKPATRICK & LOCKHART LLP, 75 STATE STREET, BOSTON, MA, 02109-1808, US

(21) Appl. No.: 10/275,233

(22) PCT Filed: May 18, 2001
(86) PCT No.: PCT/US01/16129

(51) Int. Cl. A61B 17/32

(52) U.S. Cl. 606/170; 604/95.04

(57) ABSTRACT

The present invention provides steerable biliary catheters and methods for their use. Biliary catheters such as ERCP cannulas, papillatomes, stone balloon catheters and balloon dilatation catheters may be configured to have steering capability. Two or four-way steering capability is described. Methods for performing biliary procedures such as cannulating a papilla are also provided.

### FIELD OF THE INVENTION

The present invention relates to biliary catheters having steering capability and methods for their use.

### BACKGROUND OF THE INVENTION

Biliary catheters are delivered into a patient through an endoscope previously advanced through the esophagus and into the duodenum of the patient. The catheters are advanced through the working channel of the endoscope, extend from the distal opening of the endoscope and are maneuvered across the Papilla of Vater and access the common bile duct of the biliary tree. However, navigating the distal end of a biliary catheter into the papilla can be difficult. The papilla is not easy to see and is not always oriented in a convenient location relative to the position of the endoscope in the duodenum. Side viewing endoscopes often are equipped with a movable elevator at the base of their distal opening, which is oriented to engage a catheter exiting through the port to alter the catheter's orientation and to help steer it to the intended location.

ERCP cannulas and other biliary catheters are often offered with a pre-curve distal tip to additionally help provide directional control over the distal end of the cannula as it is navigated into the papilla. Papillatome catheters have an exposed cutting wire at their distal tip, which extends parallel to the outside surface of the catheter for a short distance. The cutting wire is anchored at its distal end inside the catheter tip. However, it remains movable throughout the remainder of its length so that it may be pulled, causing the distal tip of the papillatome to arch. The movability of the cutting wire provides some steerability of the papillatome to aid in directing its distal tip into the papilla of vater. However, although the above-mentioned features help to provide some directional control over the distal end of the biliary catheter during the delicate maneuver of entering the papilla, the procedure remains a difficult one and could be aided by additional directional control over the distal end of the biliary catheter.

Steerable catheters have been used in cardiology and electrophysiology fields of medicine for some time. The steerable catheters typically comprise one or more steering cables extending the length of the catheter and contained within a lumen of the catheter that are anchored at the distal tip. The cables are pulled alternatively to cause bending in the distal tip of the catheter in the direction that tension is applied. The proximal end of the cables are typically joined to a control handle having a knob or slide that facilitates the physician's manipulation of the control wires. The resulting deflection of the distal tip of the catheter helps the physician to direct the catheter into a particular vessel at the point of intersection with another vessel. However, heretofore, biliary catheters have not been provided with the steering technology prevalent in other medical fields. It would be desirable to provide biliary catheters having steering capability and methods for their use to help cope with the difficult navigation into the Papilla of Vater from the distal end of an endoscope. Improved devices and techniques for cannulating the papilla would reduce biliary procedure time and reduce the occurrence of irritation of the papilla and the surrounding area.

### SUMMARY OF THE INVENTION

The present invention provides steerable biliary catheters and methods for their use. Biliary catheters such as ERCP cannulas, papillatomes, stone balloon catheters and balloon dilatation catheters may be provided with this steering technology.

The steering technology may be similar to that disclosed in U.S. Pat. Nos. 5,383,852 and 5,715,817, the entirety of which are incorporated by reference herein. As incorporated into biliary catheters, the disclosed steering capability provides either two-way or four-way steering capability of the distal tip of the catheter. In a two-way system, the distal tip of the catheter is deflectable in a single plane and may be thought of as being deflectable either left or right, or bi-directional. In a four-way system, the distal tip of the catheter is deflectable in two planes, 90 degrees apart and may be thought of as being deflectable, left or right and up or down, bi-directional in each direction.

As applied to a papillatome catheter, having a cutting wire capable of deflecting a distal tip, the steering system should be modified to utilize the steering capability of the cutting wire that is already present, in the catheter. The papillatome preferably incorporates a steering system for two-way directional control (in one plane) and uses the tensioning control of the cutting wire to control movement of the distal tip in a second plane. However, the cutting wire provides limited control of the tip in only one direction because a second steering cable is not provided to provide an opposing steering force on the tip for bending movement in both directions. In this sense, the steering action provided by the cutting wire may be thought of as unidirectional in that plane. It is expected that the two-way steering control mechanism, plus the limited movement of the tip provided by the cutting wire in a plane that is 90 degrees apart from the steering control plane, provides sufficient steering control for navigation in a biliary procedure. However, another single steering wire may be applied through the catheter, anchored 180 degrees apart from the cutting wire from the anchoring position of the cutting wire to provide equal steering capability in either direction along the plane defined by the cutting wire.

Additionally, with the papillatome, the steering control wires should be either non-conductive or well insulated to prevent grounding of the radiofrequency charge that is transmitted through the cutting wire to perform a papillotomy. Teflon is known to be an adequate insulator of metal wires used in catheters adjacent RF charged elements and may be applied to the control wires as a jacket or a coating. Alternatively, the lumen walls may be configured to have sufficient thickness to serve as an insulator. For Teflon, a common material for biliary catheters, a wall thickness of 0.005″ surrounding the cutting wire lumen should provide suitable insulation from the charge carried along the length of the cutting wire.

The incorporation of steering control into biliary catheters also provides for new methods for using the steerable biliary catheter in navigating to cannulate the Papilla of Vater. A method of navigating the steerable biliary catheter includes affirmatively steering the distal tip of the catheter to orient it in line with the papilla to facilitate cannulation.

It is an object of the present invention to provide biliary catheters having steering capability to facilitate cannulation of the Papilla of Vater.

It is another object of the invention to provide methods for using a biliary catheter having steering capability.

It is another object of the invention to provide a method of cannulating the Papilla of Vater.

It is another object of the present invention to provide a biliary cannula having bidirectional steering capability in a single plane.

It is another object of the present invention to provide a biliary cannula having bidirectional steering in two planes.

It is another object of the present invention to provide a papillatome catheter having bi-directional steering in one plane and unidirectional along the plane of the cutting wire.

### BRIEF DESCRIPTION OF THE DRAWINGS

The foregoing and other objects and advantages of the invention will be appreciated more fully from the following further description thereof, with reference to the accompanying diagrammatic drawings wherein:

FIG. 1 is a side view of a prior art ERCP cannula;

FIG. 2 is a side view of a prior art papillatome catheter;

FIG. 3 is a detailed sectional side view of the distal tip of a prior art papillatome;

FIG. 3A is a detailed side view of the control handle of a prior art papillatome;

FIG. 4 is a side view of a single lumen ERCP cannula with steering control;

FIG. 4A is a sectional view of the distal end of a single lumen ERCP cannula with steering control taken along the line 4A-4A in FIG. 4;

FIG. 4B is an isometric view of a control handle for a single lumen ERCP cannula with steering control;

FIG. 4C is an end view of a control handle for a single lumen ERCP cannula with steering control;

FIG. 5 is a side view of a two lumen ERCP cannula with steering control;

FIG. 5A is a sectional view of the distal end of a two lumen ERCP cannula with steering control taken along the line 5A-5A in FIG. 5;

FIG. 5B is an isometric view of a control handle for a two lumen ERCP cannula with steering control;

FIG. 5C is an end view of a control handle for a two lumen ERCP cannula with steering control;

FIG. 6 is a diagrammatic illustration of the steering capability of the 4-way steerable cannula;

FIG. 7 is a side view of a papillatome with steering control;

FIG. 8 is a diagrammatic illustration of the steering capability of the 2-way steerable papillatome;

FIG. 9 is a detailed sectional side view of the distal tip of FIG. 3A is a detailed side view of the control handle of a prior art papillatome;

FIG. 9A is a sectional view of the distal end of a papillatome taken along the line 9A-9A of FIG. 9;

FIG. 10A is a side view of a control handle for a papillatome having 2-way steering control

FIG. 11 is a diagrammatic illustration of a biliary procedure being performed using an endoscope and steerable biliary catheter.

### DESCRIPTION OF THE ILLUSTRATIVE EMBODIMENTS

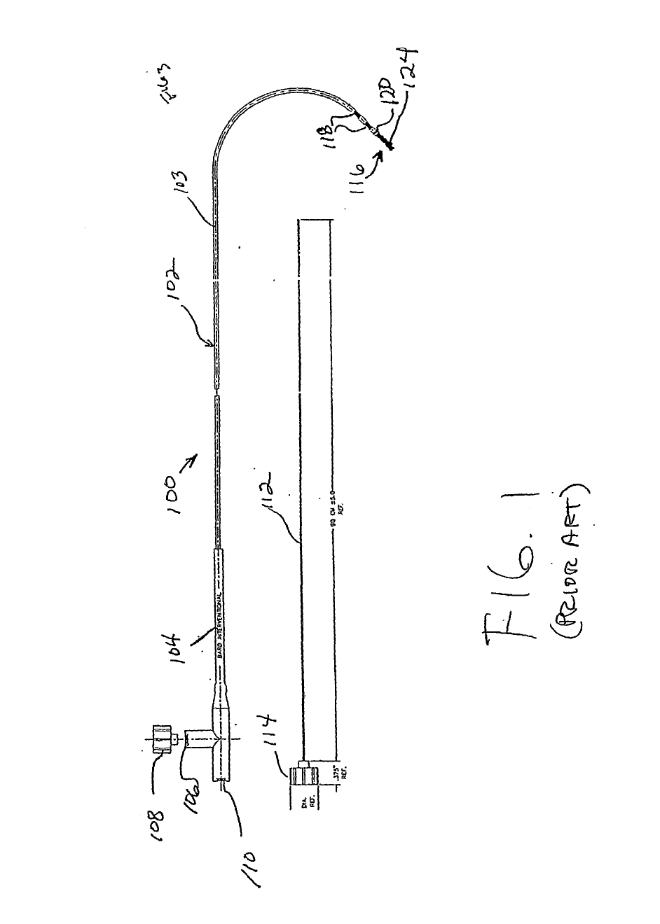

A prior art single lumen ERCP cannula product, is shown in FIG. 1. The cannula 100 comprises a single lumen Teflon tube 102 having an inside diameter of approximately 0.036 inch and a length of approximately 200 centimeters. The plastic handle 104 is over-molded to the distal end of the tube 102 for grasping by the physician during use. The handle includes a contrast media injection port 106 having a screw-on cap 108 to cover the port when not in use. The handle also has a guidewire port 110 at the proximal end of the cannula through which a guidewire may be passed. Both ports are in communication with the single lumen 103. Alternatively, a two-lumen cannula may be provided having separate lumens for the guidewire and contrast injection to provide for easier flow of the contrast and less complications with guidewire movement caused by the sticky contrast media. Three lumen cannulas are also available providing two round lumens for contrast injection and one large round lumen for the guidewire.

The cannula also may be provided with a precurved distal tip that is formed by curve retention packaging in combination with the heat to which the packaged product is exposed during the sterilization process. The preformed curve is intended to always oriented at the 12 o'clock position (the inside radius of the curve being aligned with 12 o'clock which coincides with the position of the exit port on the endoscope). That orientation permits the cannula to protrude from the side exit port of the endoscope in a direction that is perpendicular to the longitudinal axis of the scope and is directed to the papilla. However, the curve is not adjustable after placement in the body and no active control mechanism exist in the cannula prior art, of which applicants are aware that permit redirection of the cannula distal tip from the proximal (control) end of the device.

A guidewire may, but need not, be first inserted through the cannula in order to deliver it through the endoscope and into the papilla. Though a guidewire is useful to provide column strength to advance the cannula through the endoscope, the distal tip of a guidewire extending from the distal end of cannula would tend to prolapse, if highly flexible, or irritate the papilla, if stiff. Therefore, it is preferable to keep the guidewire retracted inside the tip of the cannula during delivery through the papilla. As an alternative to this practice, a separable short stiffening wire 112 is provided with the cannula. The stiffening wire extends partially through lumen 103 to provide axial support in the proximal regions of the cannula in the absence of a guidewire during delivery through an endoscope. The stiffening wire has a length of approximately 90 centimeters, only about half the length of the cannula and may be securely fastened within the cannula by screw-on cap 114 joined to the proximal end of the wire and threadably engageable with the guidewire port 110. Once the cannula has been delivered into the papilla, the stiffening wire 112 may be removed and a guidewire inserted through the lumen 103 and into the common bile duct, where it can remain throughout the procedure as a track over which subsequently selected instruments may be introduced.

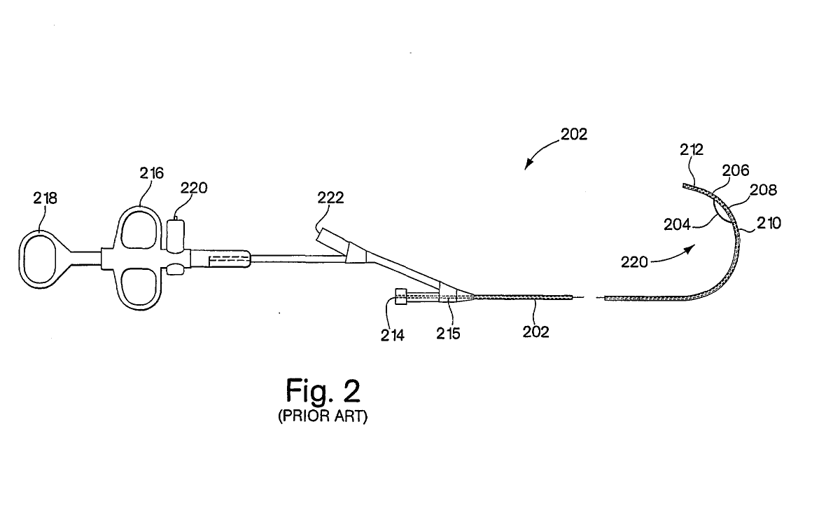

A prior art papillatome is shown in FIG. 2. The papillatome 200 is a multilumen catheter having a radio frequency energizable external cutting wire at its distal end that is used to make a radially extending cut through the annular shaped Papilla of Vater. Cutting the papilla is intended to expand the opening that it defines to then permit stones trapped in the common bile duct to pass freely into the duodenum. The papillatome 200, comprises a two or three lumen Teflon shaft 202. At the distal end of the shaft, an external cutting wire 204 extends parallel to the catheter for a short distance. Underlying the exposed portion of the wire are several positioning bands 206, 208 and 210 and a distal band 212 at the distal tip of the catheter to provide a visual indication to the physician of how far the tip has entered the papilla, such bands may also be found on the cannula described above.

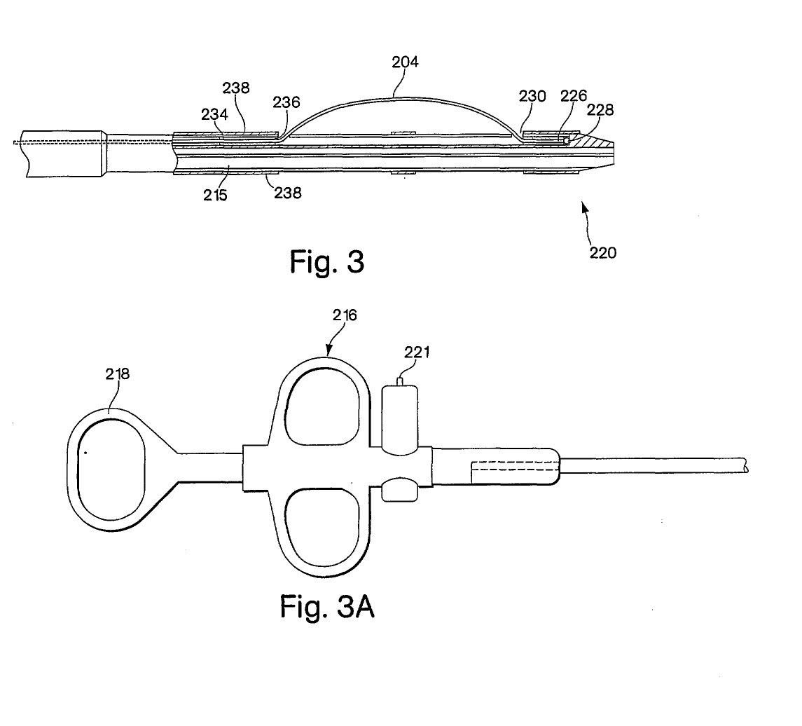

At the proximal end of the catheter, a guidewire port 214 opens to a guidewire lumen 215 extending through the length of the catheter. Either a full length guidewire or a shortened stiffening wire (as with the cannula) may be used in the guidewire lumen of the papillatome. As shown in detail in FIG. 3A, a handle 216 with thumb slide 218 is joined to the cutting wire and cutting wire lumen at the proximal end of the papillatome to control operation of the cutting wire 204. The thumb slide is joined to the proximal end of the cutting wire and can be slid proximally to pull the cutting wire tout to begin cutting the papilla. Pulling the cutting wire tout also serves to deflect the distal tip of the papillatome slightly along the plane of the cutting wire. Releasing the tension on the cutting wire permits the distal tip to relax back to its undeflected orientation. This limited range of distal tip movement provides some steering control, but only in one direction and along one plane.

As shown in FIG. 3A, a connector 221 may be joined to a source of radio frequency energy to energize the cutting wire 204, which is electrically insulated as it extends through an independent second lumen of the catheter. Contrast media may be injected through the guidewire lumen as needed during the procedure. Optionally, a third lumen open to injection port 222 may be provided for the injection of contrast media into the biliary tree once the distal end 220 of the papillatome has been inserted through the papilla.

A detailed drawing of the distal tip 220 of the papillatome 200 is shown in FIG. 3. The cutting wire 204 extends external of the catheter shaft along a portion of the distal tip. The distal end 226 of the cutting wire enters the catheter at distal exit port 230 and is anchored in a cutting wire lumen 234 of the catheter, near the distal tip, at anchor joint 228. Extending proximally, the cutting wire 204 is parallel and external to the distal tip of the catheter for approximately 0.78 inch, reentering the cutting wire lumen 234 at proximal port 236. The wire enters a collar 238 wedged in the cutting wire lumen just proximal to the proximal port 236. The collar provides structural support to the lumen in this area to resist pulling and tearing caused by movement of the cutting wire. The cutting wire 204 extends approximately 12 inches proximal from the distal tip before being joined to a separate round wire of approximately 0.020 inch in diameter which then continues proximally to the thumb slide 218 in the handle 216. The cutting wire is joined to the round wire by a collet to which both wires are braised.

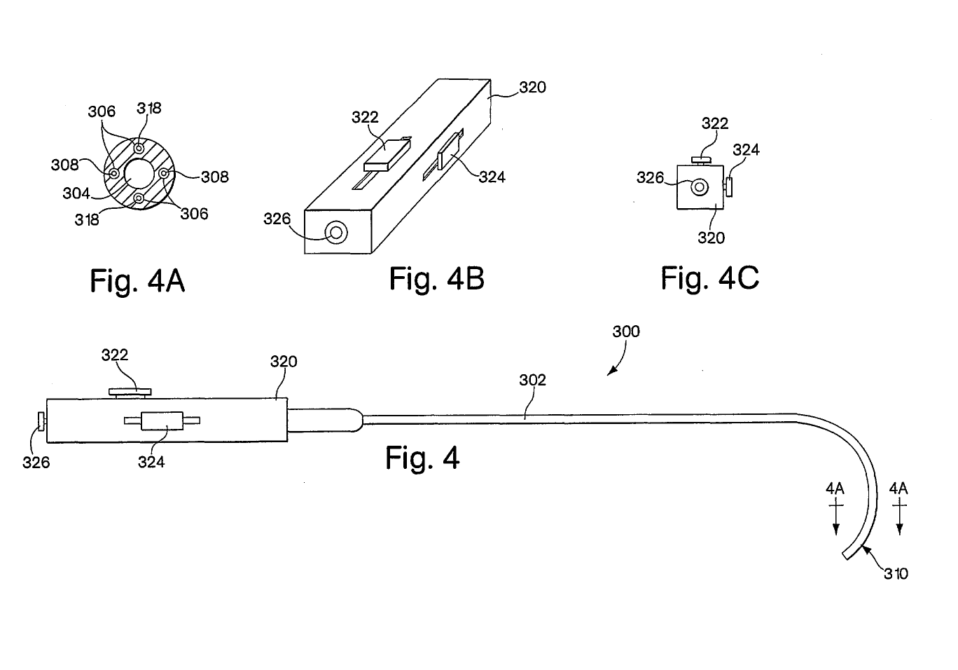

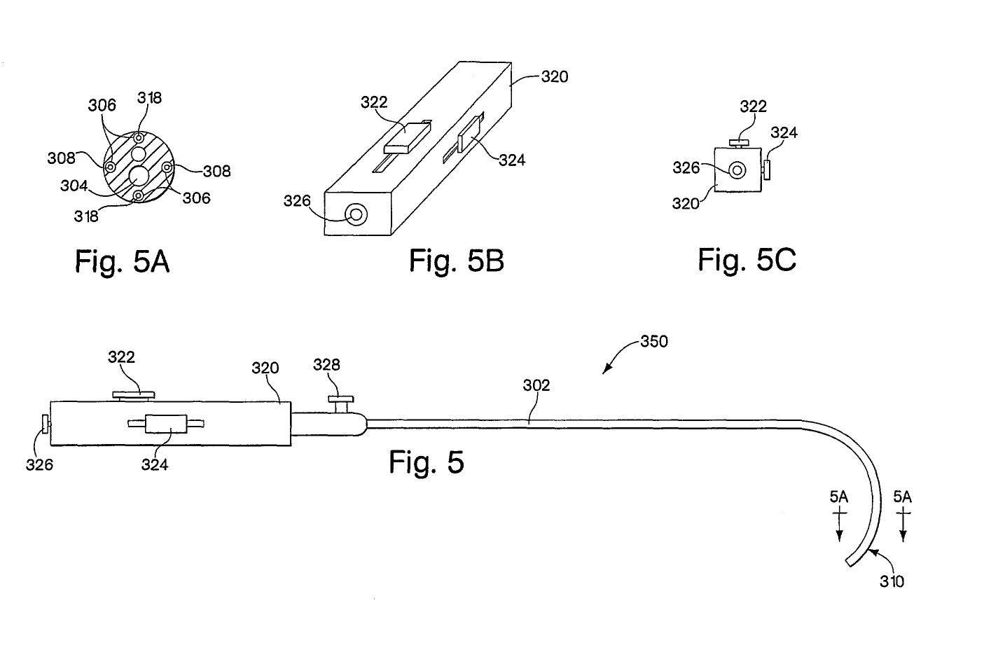

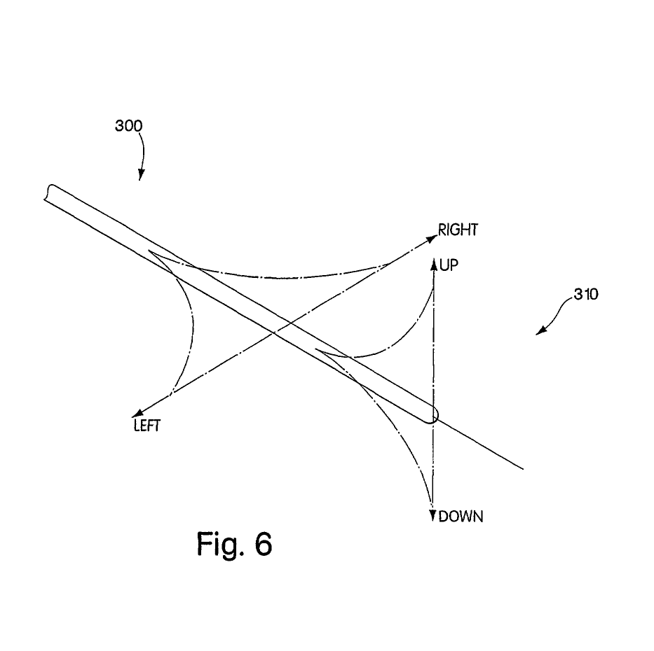

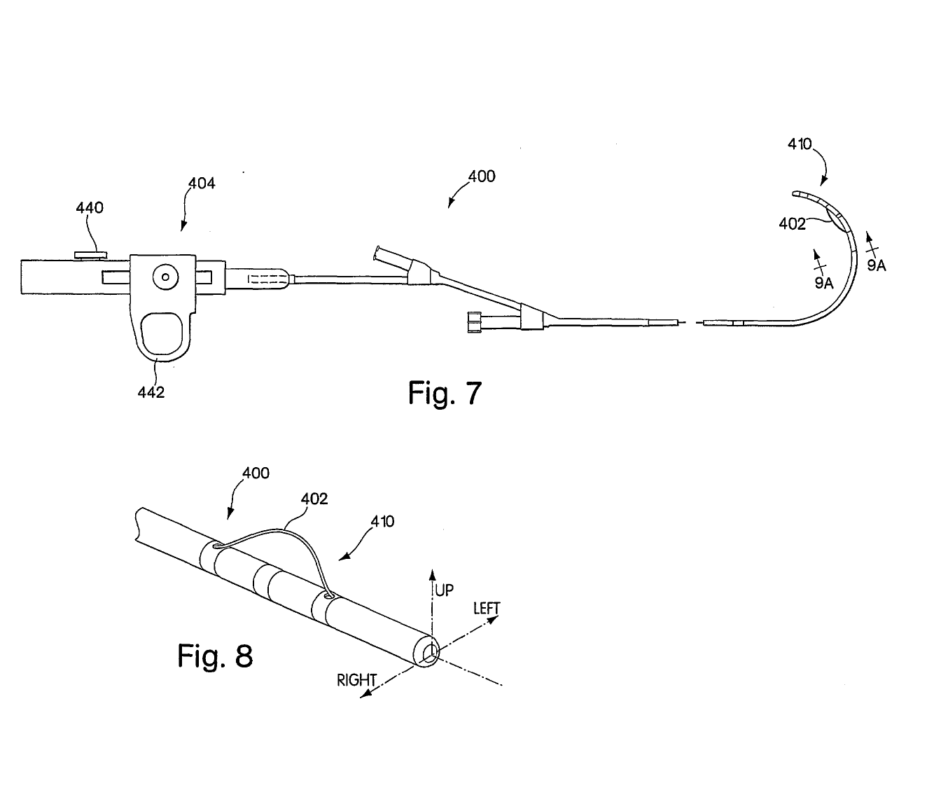

FIGS. 4-4C show a steerable, single lumen cannula 300 according to the present invention. The cannula comprises a shaft 302 having one main lumen 304 shared for guidewire placement and contrast injection. The shaft of the steerable cannula also includes small steering wire lumens 306, equally circumferentially spaced around the main lumen 304. The steering wire lumens slidably receive steering wires 308 and 318 anchored in the lumens adjacent the distal end 310.

The wires are arranged to be placed in tension to cause deflection of the distal tip in the direction of the tightened wire. In a two way system two steering wires 308 and lumens 306 are arranged in the shaft 302 diametrically opposed, 180 degrees apart. In a four way steering system four steering wires (two sets of two) 308 and 318 and four lumens 306 are provided, equally spaced, 90 degrees apart as shown in FIG. 4A.

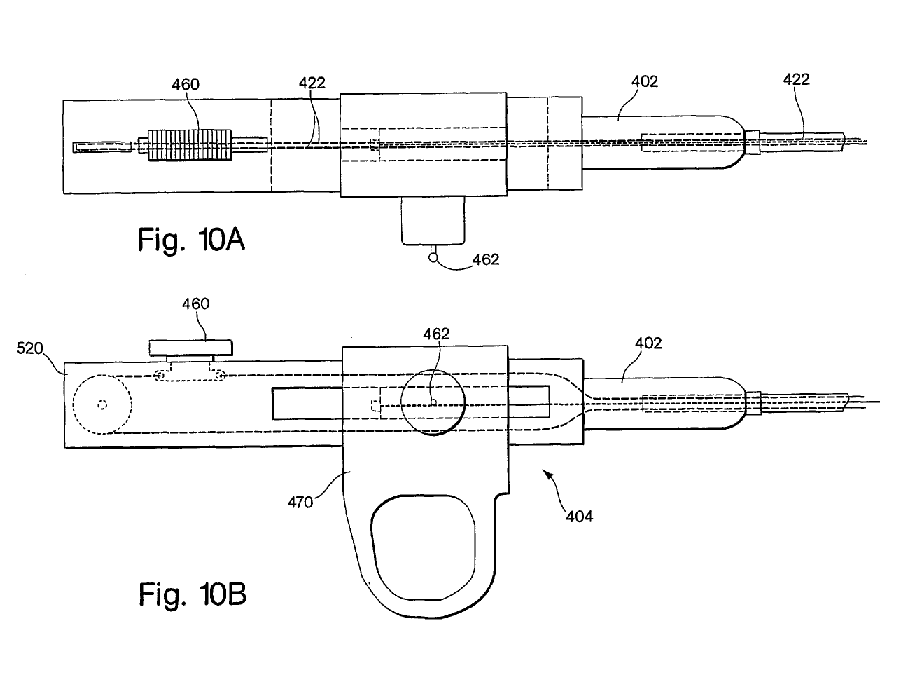

Joined to the proximal end of the steerable cannula is a control handle 320. The control handle provides steering controls 322 and 324 that are joined to the steering wires 308 and 318. The handle also has a port 326 through which the guidewire may be passed or contrast injection inserted. An example of the connections between the controls and the steering wires is shown in FIGS. 10A and 10B. The controls may comprise slides joined to a portion of the wire. Each wire is U-shaped in that its free ends are anchored in the lumens adjacent the distal end 310 and the proximal end is supported by a pulley 520 supported by the interior of the handle.

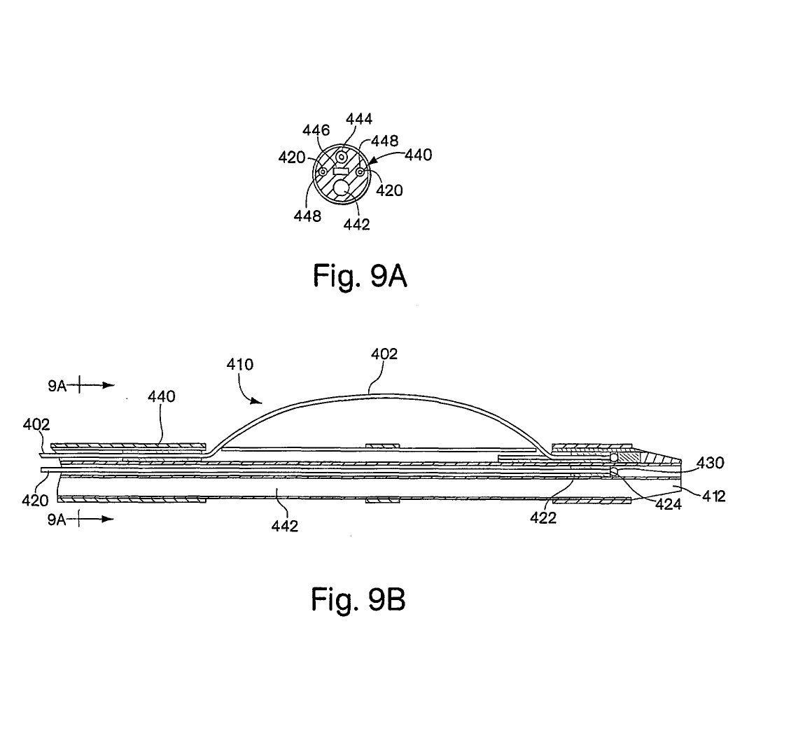

An example of anchoring wires in the distal end is shown in the example of a papillatome catheter in FIG. 9. The ends of the wires 420 may be anchored by adhesives. Preferably however, the wires are anchored by small reinforcing segments of hypotubing 422 embedded into the lumen by application of heat during a neckdown procedure of the distal end 410 through a hot die. The wire distal ends 424, loaded from the proximal end of the catheter may be extended through the distal tip 412 of the catheter, welded to have an oversized ball 430 formed on the end, then pulled 446 is located between the cutting wire lumen and guidewire lumen. To accommodate the small space left between those two lumens, the contrast media lumen 446 may be of a rectangular cross section, on the order of 0.006 by 0.020 inches. Steering wire lumens 448 are located, diametrically opposed at the 3:00 and 9:00 positions of the shaft cross section. The arrangement and connection of the steering wires 422 are as explained above in connection with the cannula.

The steering control for the papillatome is provided through use of the control handle 404 shown in FIGS. 10A and 10B. The control handle comprises a steering control slide 460 joined to steering control wire 422 inside the control handle 404. The operation of the steering control slide 460 and the control wire 520 is explained above in connection with the cannula. The papillatome control handle additionally provides a cutting wire control slide 470, the sliding movement of which controls longitudinal movement of the cutting wire. Because the cutting wire is anchored at the distal end 410 of the papillatome, movement of control 470 serves to create or release tension in the cutting wire that deflects the distal tip 410 slightly in one direction to provide some steering control. Additionally, the cutting wire control slide 470 provides a electrical connection 462 for receiving RF current to the cutting wire. The handle may be joined to the shaft 410 by conventional methods known in the art.

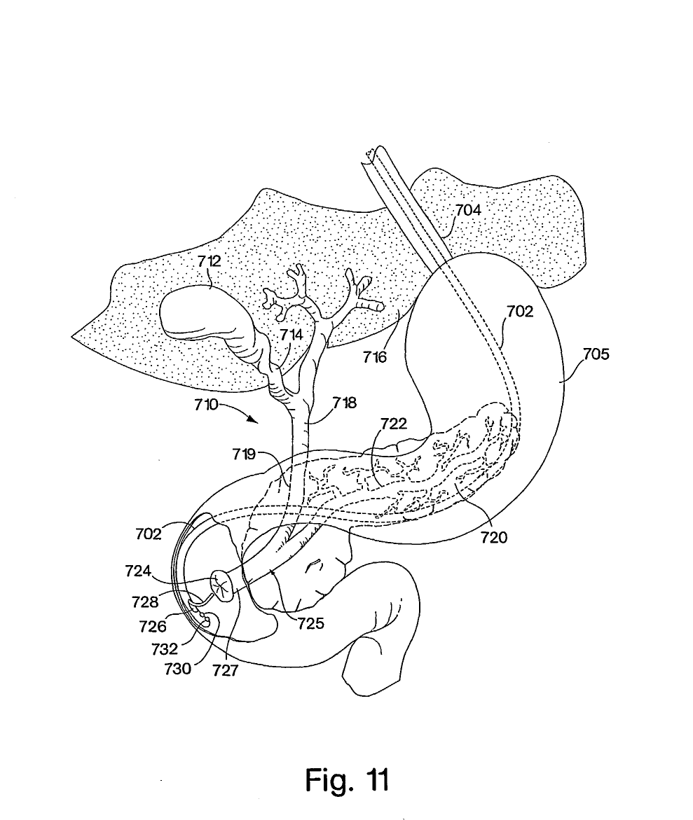

FIG. 11 is a detailed illustration of an endoscopic biliary procedure being performed with a steerable biliary catheter. An endoscope 702 having viewing capability and a working channel is first navigated down the esophagus 704 of a patient. The endoscope is advanced through the stomach 705 and into the duodenum 706 at the bottom of the stomach. The biliary tree, 710 comprises the cystic duct 714 from the gall bladder 712, the hepatic duct 718 from the liver 716 and the pancreatic duct 722 from the pancreas 720. Each of these ducts joins the common bile duct 719. The common bile duct 719 intersects with the duodenum a slight distance below the stomach. The papilla (sphincter muscle) 724 controls the size of the opening at the intersection between the bile duct 719 and duodenum 706. The shared portion of the common bile duct 718 and pancreatic duct 722 extending from the papilla 724 is known as the Ampulla of Vater 725. Muscle entwined with Ampulla of Vater duct serves to selectively constrict the duct to control the flow bile and pancreatic secretions. The constricting muscle is known as the Sphincter of Oddi 727.

The papilla 724 must be crossed by the biliary device 728 in order to reach the common bile duct to perform a biliary procedure. Without steering capability in the biliary catheter, the endoscope must be positioned carefully if the papilla is to be crossed by the catheter. The endoscope is navigated so that the side exit port 726 is directly across from the papilla (short endoscope position) so that when the biliary device exits the sideport of the endoscope and extends perpendicular to the endoscope, it is aligned to enter the papilla. However, steering capability in the catheter 728 makes alignment with the papilla easier, regardless of the scope position because the distal tip of the catheter can be maneuvered to meet the papilla during advancement.

After positioning the endoscope so that the side port 726 of the working channel is adjacent the papilla 724, the steerable biliary catheter 728 is advanced through the working channel of the endoscope such that the distal end of the steerable biliary catheter emerges from the side port 726 of the endoscope. Side viewing port 730 and light 732 of the endoscope are arranged to provide viewing of the catheter 728 as it emerges from the endoscope and is moved to enter the papilla 724. Typically, an ERCP cannula is first navigated into the papilla. A guidewire, or partial length stiffening element is preloaded into a lumen of the cannula, but does not protrude from its distal end during navigation through the papilla. The catheter tip is then steered to be in alignment with the papilla, then the catheter is advanced so that it penetrates the papilla. After crossing the papilla, the cannula advanced into the common bile duct along with the guidewire. After the initial cannulation, the guidewire is left in place in the common bile duct to provide a track over which other biliary devices may be easily introduced.

ERCP cannulas and other biliary catheters are offered with a precurved distal tip intended to facilitate alignment of the tip when exiting from the side exit port 726 of the endoscope and approaching the papilla. However if the catheter is not rotated correctly in the scope or if the scope is slightly mispositioned, only the steering capability of the distal tip of the biliary catheter can provide navigational options to the physician for reaching the papilla easily. Additionally, a moveable heel mechanism at the distal side of the exit port 726 of the endoscope helps to provide additional steering capability by adjusting the orientation of the exiting biliary device. Combining the steering capability of the catheter with the heel mechanism gives an exceptional range of directional during a biliary procedure. The distal end of the biliary device preferably has a series of visual markers, spaced approximately 3 millimeters apart, to aid the physician in determining whether the distal tip of the device has entered the papilla a sufficient amount so that a contrast media may be injected to visualize the biliary tree.

After the biliary tree has been visualized radiographically, the physician can diagnose the problem afflicting the biliary system and, with other specialized catheters and devices inserted through the endoscope, treat the particular ailment. Common ailments of the biliary system include gallstones or strictures in the various ducts of the biliary tree. Biliary catheters such as stone balloon catheters and dilatation balloon catheter benefit from steerable configurations as exemplified in the cannula and papillatome embodiments discussed above, even if introduced over an indwelling guidewire because they can be steered into the various ducts that branch from the common bile duct if treatment so requires.

In the case of stones in the common bile duct 719, the physician may attempt to permit them to pass from the duct by enlarging the opening of the papilla 724 with a papillatome cutting catheter. If the papillatome is the first used instrument in the procedure, steering control in addition to that provided by the cutting wire, facilitates locating and entering the papilla as was demonstrated above in the discussion of the cannula

It should be understood however, that the foregoing description of the invention is intended merely to be illustrative thereof and that other modifications, embodiments and equivalents may be apparent to those who are skilled in the art without departing from its spirit.

Having thus described the invention what we desire to claim and secure by letters patent is:

1. A steerable biliary catheter comprising:
a flexible shaft having at least one lumen, proximal and distal end;
a plurality of control wires extending through the catheter, anchored at the distal tip, such that tension applied to the wires causes pulling and deflection of the tip;
a control handle connected to the control wires to effect manipulation of the wires through the catheter.

2. A steerable papillatome catheter comprising:
a flexible elongate shaft having at least one lumen;
a cutting wire conductive of radiofrequency energy extending through the lumen of the shaft, anchored at the distal tip of the shaft and extending exterior of the shaft for a short distance near the distal end;
a plurality of control wires each extending through at least one lumen of the shaft;
a control handle joined to the control wires in an operable connection that permits longitudinal movement of the control wires when the handle is operated;
insulation applied between the control wires and the cutting wire element.

3. A catheter as in claims 1 or 2 wherein a sufficient number of control wires are provided to provide steering control at the distal tip in two directions.

4. A catheter as defined in claims 1 or 2 wherein an adequate number of control wires are provided to provide steering control of the distal tip in four directions.

5. A method of cannulating the papilla of vater comprising:
providing an endoscope;
providing a steerable biliary catheter having steering control of the distal tip in two directions;
navigating the endoscope adjacent to the papilla of vater;
inserting the steerable catheter through the endoscope to exit the distal port of the endoscope;
steering the distal tip of the catheter along the plane defined by the two directions of steerability and advance the catheter to enter the papilla.

6. A method of cannulating the papilla of vater comprising:
providing a side viewing endoscope;
providing a steerable biliary catheter having steering control in four directions;
navigating the endoscope through the esophagus and duodenum so that its distal end is adjacent the papilla;
introducing the catheter through the endoscope and advancing it until the distal end of the catheter protrudes from the exit port of the endoscope;
advancing the catheter and steering the distal tip along the two planes defined by the four directions of steerability to enter and cannulate the papilla.
